(19) United States
(12) Patent Application Publication
(10) Pub. No.: US 2026/0144475 A1
(43) Pub. Date: May 28, 2026

(54) Body Surface Gastric Mapping Artifact Removal and Correction Methods and Systems

(71) Applicant: Alimetry Limited, Auckland (NZ)

(72) Inventors: Nicole H. Dachs, Auckland (NZ); Armen A. Gharibans, Auckland (NZ); Gregory B. O'Grady, Auckland (NZ); Gabriel Schamberg, Auckland (NZ)

(73) Assignee: Alimetry Limited, Auckland (NZ)

(21) Appl. No.: 19/400,681

(22) Filed: Nov. 25, 2025

Related U.S. Application Data
(60) Provisional application No. 63/724,781, filed on Nov. 25, 2024.

(51) Int. Cl. A61B 5/397; A61B 5/00; A61B 5/296; A61B 5/392; G16H 50/20

(52) U.S. Cl. CPC A61B 5/397; A61B 5/296; A61B 5/392; A61B 5/7203; A61B 5/7246; A61B 5/7267; G16H 50/20

(57) ABSTRACT

Methods and systems of processing gastric activity data according to the present disclosure include receiving electrical signals associated with gastric activity of a patient over a predetermined time period. The electrical signals are measured with an electrode array patch disposed over an abdomen skin surface of the patient. For each received signal, the method includes providing the signal as an input signal to a correction neural network and a removal neural network, and outputting, by the correction neural network, an intermediate corrected signal generated based on the input signal. The method includes generating an initial report based on the intermediate corrected signal where the initial report includes a gastrointestinal phenotype based at least in part on the intermediate corrected signal.

### CROSS REFERENCES TO RELATED APPLICATIONS

The present application claims the benefit of priority to U.S. Provisional Appln. No. 63/724,781 filed Nov. 25, 2024, the full disclosure which is incorporated herein by reference in its entirety for all purposes.

### BACKGROUND

Chronic gastro-duodenal symptoms affect more than 10% of the global population and have a significant healthcare burden, resulting in a significant economic impact. Functional gastrointestinal (GI) disorders are among the most prominent causes of chronic ill-health in both adults and children. Chronic gastroduodenal diagnosis paradigms rely on symptom-based criteria which group nausea, vomiting, abdominal pain, early satiety, and/or excessive fullness into disorders such as chronic nausea and vomiting syndromes (CNVS), functional dyspepsia (FD), and when gastric emptying is delayed, gastroparesis. However, these classifications substantially overlap, limiting their clinical utility and ability to effectively inform individual patient management.

Functional gastrointestinal disorders (FGIDs, or disorders of gut-brain interaction) place an economic burden on healthcare systems and reduce patient quality of life. Functional gastrointestinal disorders generally affect 35% to 70% of people at some point in life, women more often than men. For example, more than 70% of patients indicate that their symptoms interfere with everyday life and 46% report missing work or school. A recent review of 26 studies found that between 10-29% of school children reported symptoms consistent with a functional GI disorder. The symptoms are frequently distressing and may be severe and debilitating, encompassing chronic abdominal pain, abdominal distension, anorexia, and chronic nausea and vomiting. These disorders collectively extract a major illness burden, including a significantly reduced quality of life, and are common reasons for adults and children missing work or school.

Current GI diagnoses include gastroparesis and functional dyspepsia disorders. Gastroparesis is defined by symptoms of nausea and vomiting, typically with other symptoms e.g., abdominal pain, bloating, burning, excessive fullness, early satiation, and/or documented presence of delayed gastric emptying. Functional dyspepsia is defined by chronic symptoms such as distress after eating, indigestion, abdominal pain, bloating, burning, excessive fullness, and/or early satiation. Gastric emptying may also be delayed in up to 25% of patients identified with functional dyspepsia, and therefore overlaps with gastroparesis, however nausea and vomiting are not considered the dominant feature. Because these disorders overlap significantly, or at least many patients are on the same disease spectrum, there is a state of confusion in the clinical field. For example, clinicians are often unsure how to define, distinguish and diagnose such patients, and therefore are unable to provide appropriate patient specific management plans, typically reverting to trial and error type therapies.

Objectively evaluating and treating adults and children with chronic upper GI symptoms is a major clinical challenge, owing to a lack of routine tests that may reliably and safely distinguish specific underlying disorders. Relying on symptom-based diagnoses often results in less than ideal and potentially hazardous attempts at trial-and-error treatments. Currently, both adult and pediatric patients with chronic GI symptoms frequently undergo a protracted diagnostic process that may include endoscopies, biopsies, lab tests, nuclear medicine studies, manometry and radiology exams, often over numerous years. Many of these tests are invasive and involve radiation, yet the diagnostic results are often inconclusive. For example, gastric scintigraphy and antroduodenal manometry are two tests that are commonly performed in adult and pediatric gastroenterology, as they may distinguish myopathic or neuropathic functional disorders, and may impact diagnosis and treatment in 15-20% of patients with chronic upper GI symptoms. However, the interpretation of these tests may be uncertain, especially in pediatric applications due to a lack of diagnostic norms in children. Furthermore, these tests typically involve long wait times and high cost as these tests are generally only available in specialist referral centers.

There is a pressing need for improved and less invasive diagnostic tests that have clinical utility, offer actionable and objective biomarkers that improve both adult and pediatric diagnostic and treatment efficacy, reduce patient harm from negative invasive or unnecessary testing, and directly impact clinical care decisions and treatment. The advent of a less invasive and technically safer diagnostic test for adults and children would broaden availability and access and reduce the high healthcare expenses of motility testing. An optimal diagnostic solution would be non-invasive, user-friendly, easy to apply and interpret, and provide meaningful results that correlate with symptoms and inform clinical care.

### SUMMARY

The present invention is directed to user-friendly methods and systems for mapping gastric activity for objective symptom profiling and gastrointestinal phenotyping, thereby providing efficacious and reliable diagnosis and appropriate therapeutic options for both adult and pediatric patients. Various embodiments of the present invention include non-invasive gastric activity detection systems, such as an electrode array patch and data acquisition/connector device for mapping gastric activity. Embodiments described herein may be used in the diagnosis and therapy of adults and children presenting with functional upper GI symptoms by monitoring, analyzing and optimizing measured electrical signals from the non-invasive electrode array patch to provide meaningful results that correlate with symptoms and inform clinical/patient care.

Embodiments include utilizing real-time patient reported symptoms as a component of the gastrointestinal system for clinical assessment and diagnosis of gastro-duodenal disorders. Embodiments of the present invention have been shown to provide superior results over the Rome diagnostic questionnaire. At least some embodiments of the present invention advantageously avoid the classification of the presence or absence of symptoms over a long timeframe (typically months) and/or avoid significant overlap between symptoms and/or diagnoses (e.g., gastroparesis, functional dyspepsia, and/or chronic nausea and vomiting) and/or provide insight into patient specific symptom etiology and appropriate patient specific treatment plans.

Various embodiments are directed to identifying abnormal gastric motility in a significant subgroup of patients with chronic gastric conditions. In some embodiments, the teachings herein are directed to identifying gastric motility that affects only a subset of people falling within a group in the overall population that are less than, greater than, or equal to 1, 2, 3, 4, 5, 6, 7, 8, 9, 10, 11, 12, 13, 14, 15, 16, 17, 18, 19 or 20% or any value or range of values therebetween in 1% increments. Embodiments of the present invention provide a reliable and objective method for assessing gastric motor function in clinical practice. Embodiments have been shown to provide correct assessment at least a 75, 80, 85, 90, 91, 92, 93, 94, 95, 96, 97, 98 or 99% or greater rate or any value or range of values therebetween in 1% increments out of at least 50, 100, 200, 300, 400 or 500 or more random patients.

At least some of the embodiments described herein provide a standardized system for quantitative assessment of an individual patient. The system may include continuous or semi-continuous assessment of symptom severity particularly after a meal stimulus for the purposes of diagnostic data collection. Systems described herein use Body Surface Gastric Mapping (BSGM) employing multi-electrode array patches, as described in further detail in U.S. Patent Application No. US 2023-0083795 A1, which is incorporated herein by reference in its entirety for all purposes, to measure and map gastric myoelectrical activity. BSGM may be used in some embodiments of the presentation invention to provide high-quality and high-resolution information non-invasively. Embodiments may also include semi-automated digital and/or analogue tools developed for receiving standardized gastric symptom profiling. These tools may also be used simultaneously during testing to further aid in the identification/refinement of specific disease phenotypes.

The present invention relates to a body surface electrode mapping assembly and a method of manufacture of the same. The body surface electrode mapping assembly includes a flexible substrate that conforms to a patient's skin while taking measurements and includes a stiffener layer that prevents unwanted stretching and/or deformation during storage, transport, manufacturing, placement, etc. The body surface electrode mapping assembly may be interchangeably referred to herein as an electrode patch assembly. The body surface electrode mapping assembly may be positioned on an abdomen of a patient for sensing and mapping of gastric or colonic electrical signals. Mapping gastric or colonic electrical signals may include various embodiments as described in U.S. Provisional Application Ser. No. 63/642,417 filed May 3, 2024, entitled, “Gastrointestinal Diagnostic Aid,” and U.S. Provisional Application Ser. No. 63/648,594 filed May 16, 2024, entitled, “Systems and Methods for Body Surface Colonic Mapping,” each of which is incorporated herein by reference in its entirety for all purposes.

A method of processing gastric activity data according to the present disclosure includes receiving electrical signals associated with gastric activity of a patient over a predetermined time period. The electrical signals are measured with an electrode array patch disposed over an abdomen skin surface of the patient. For each received signal, the method includes providing the signal as an input signal to a correction neural network and a removal neural network, outputting, by the correction neural network, an intermediate corrected signal generated based on the input signal, where, in the intermediate corrected signal, each segment of the input signal identified by the correction neural network to contain noisy data is replaced with a corrected segment generated by the correction neural network, and generating an initial report based on the intermediate corrected signal where the initial report includes a gastrointestinal phenotype based at least in part on the intermediate corrected signal.

The method may include various optional embodiments. The method may further include outputting a confidence score for each segment of the input signal identified by the removal neural network to contain noisy data where the confidence score represents an estimated difference between the true noise-free signal and a predicted corrected signal. The method may further include determining whether each confidence score associated with a corrected segment is below a preconfigured threshold value. The method may further include, in response to determining that the confidence score associated with the corrected segment is below the preconfigured threshold value, replacing, by a removal module, the corrected segment with a segment representing deleted data to generate a final corrected signal, and generating a final report based on the final corrected signal, wherein the final report identifies the gastrointestinal phenotype based at least in part on the final corrected signal. The final report may include a visual representation of the final corrected signal. The visual representation may identify data segments replaced by corrected segments. The visual representation may identify data segments replaced by segments representing deleted data. The gastrointestinal phenotype may include a dysrhythmic, high frequency, low meal response, sensorimotor, continuous, or delayed onset phenotype. The method may include determining one or more normalized biometrics over at least a predetermined time period from the final corrected signal, correlating the one or more normalized biometrics and patient symptom information received over the predetermined time period, determining a measure of correlation over the predetermined time period, and determining the gastrointestinal phenotype based at least in part on the measure of correlation. The one or more normalized biometrics may include at least one of a principal gastric frequency (PGF), a body mass index (BMI)-adjusted amplitude, Gastric Alimetry Rhythm Index (GA-RI), fed-fasted amplitude ratio (ff-AR), and a meal response ratio. The method may include outputting a recommendation based at least in part on the gastrointestinal phenotype associated with the patient. The method may include training the correction neural network where training the correction neural network includes providing an original set of patient data, generating training data based on the original set of patient data, and using a training module with the training data based on the original set of patient data. The method may include generating synthetic data and training a training module using the training data based on the original set of patient data and the synthetic data. The method may include identifying segments of the original set of patient data to be classified as clean data segments and noisy data segments. The method may include generating the synthetic data, including synthesizing clean data segments and noisy data segments. The method may include augmenting and recombining the clean data segments and the noisy data segments identified from the original set of patient data. The method may include augmenting and recombining the synthesized clean data segments and the synthesized noisy data segments. The method may include augmenting and recombining the synthesized clean data segments and the clean data segments. The method may include augmenting and recombining the noisy data segments and the synthesized clean data segments. The method may include augmenting and recombining the clean data segments and the synthesized noisy data segments. The method may include augmenting and recombining the synthesized noisy data segments and the noisy data segments. The method may include training the removal neural network based at least in part on the correction neural network.

A system for processing gastric activity data according to the present disclosure includes an electrode array patch disposed over an abdomen skin surface of a patient for measuring electrical signals associated with gastric activity of the patient over a predetermined time period and a processor configured to receive electrical signals from the electrode array patch and for each received signal provide the signal as an input signal to a correction neural network and a removal neural network, and output, by the correction neural network, an intermediate corrected signal generated based on the input signal, where, in the intermediate corrected signal, each segment of the input signal identified by the correction neural network to contain noisy data is replaced with a corrected segment generated by the correction neural network, and generate an initial report based on the intermediate corrected signal, wherein the initial report includes a gastrointestinal phenotype based at least in part on the intermediate corrected signal.

A non-transitory computer-readable medium storing instructions executable by one or more processors for causing the one or more processors to perform operations according to the present disclosure includes receiving electrical signals associated with gastric activity of a patient over a predetermined time period where the electrical signals are measured with an electrode array patch disposed over an abdomen skin surface of the patient and, for each received signal, providing the signal as an input signal to a correction neural network and a removal neural network, and outputting, by the correction neural network, an intermediate corrected signal generated based on the input signal, where, in the intermediate corrected signal, each segment of the input signal identified by the correction neural network to contain noisy data is replaced with a corrected segment generated by the correction neural network. The operations include generating an initial report based on the intermediate corrected signal, where the initial report includes a gastrointestinal phenotype based at least in part on the intermediate corrected signal. The operations include outputting a confidence score for each segment of the input signal identified by the removal neural network to contain noisy data, wherein the confidence score represents an estimated difference between the true noise-free signal and a predicted corrected signal, determining whether each confidence score associated with a corrected segment is below a preconfigured threshold value; in response to determining that the confidence score associated with the corrected segment is below the preconfigured threshold value, replacing, by a removal module, the corrected segment with a segment representing deleted data to generate a final corrected signal, and generating a final report based on the final corrected signal, wherein the final report identifies the gastrointestinal phenotype based at least in part on the final corrected signal.

Other embodiments and variations thereof will become apparent from the following description which is given by way of example only and with reference to the accompanying drawings.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning, allowing for inclusion of not only the listed components or elements, but also other non-specified components or elements. The terms ‘comprises’ or ‘comprised’ or ‘comprising’ have a similar meaning when used in relation to the system or to one or more steps in a method or process.

As used hereinbefore and hereinafter, the term “and/or” means “and” or “or”, or both. As used hereinbefore and hereinafter, “(s)” following a noun means the plural and/or singular forms of the noun. As used hereinbefore and hereinafter, the term “continuous” or “semi-continuous” with respect to the test period is to be interpreted as ongoing throughout the entire or nearly entire test period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will now be described by way of example only and with reference to the drawings in which:

FIG. 1 is a perspective view of a flexible electrode patch, according to various embodiments of the present disclosure.

FIG. 2 is an exemplary comparison of a visual representation of electrical signals before and after noise correction, according to various embodiments of the present disclosure.

FIG. 3 is a schematic of a signal correction system, according to various embodiments of the present disclosure.

FIG. 4 is a flowchart of a method of processing gastric activity data, according to various embodiments of the present disclosure.

FIG. 5 is a schematic of a signal correction sub-system for training a correction neural network, according to various embodiments of the present disclosure.

FIG. 6 is a flowchart of a method of training a correction neural network, according to various embodiments of the present disclosure.

FIG. 7A is a schematic of a correction neural network training paradigm, according to various embodiments of the present disclosure.

FIG. 7B is a schematic of a correction neural network training paradigm, according to various embodiments of the present disclosure.

FIG. 8A is a schematic of a signal correction sub-system for training a removal neural network, according to various embodiments of the present disclosure.

FIG. 8B is a schematic of a signal correction sub-system for training a removal neural network, according to various embodiments of the present disclosure.

FIG. 9 is a flowchart of a method of training a removal neural network, according to various embodiments of the present disclosure.

FIG. 10A is a schematic of a removal neural network training paradigm, according to various embodiments of the present disclosure.

FIG. 10B is a schematic of a removal neural network training paradigm, according to various embodiments of the present disclosure.

FIG. 10C is a schematic of a removal neural network training paradigm, according to various embodiments of the present disclosure.

FIG. 11 is an exemplary report of gastric activity data, according to various embodiments of the present disclosure.

FIGS. 12A-12B illustrate an exemplary comparison of a visual representation of electrical signals before and after noise correction, according to various embodiments of the present disclosure.

FIG. 13 is an exemplary signal quality report, according to various embodiments of the present disclosure.

FIG. 14 illustrates exemplary processed signals, according to various embodiments of the present disclosure.

FIG. 15 illustrates exemplary spectrograms and accelerometer-based activity, according to various embodiments of the present disclosure.

FIG. 16 illustrates exemplary spectrograms and accelerometer-based activity, according to various embodiments of the present disclosure.

For purposes of the description hereinafter, the terms “upper”, “lower”, “right”, “left”, “vertical”, “horizontal”, “top”, “bottom”, “lateral”, “longitudinal” and derivatives thereof shall relate to the teachings herein as it is oriented in the drawing figures. However, it is to be understood that the variations of the teachings herein may assume various alternative variations, except where expressly specified to the contrary. It is also to be understood that the specific devices illustrated in the attached drawings and described in the following description are simply exemplary embodiments. Hence, specific dimensions and other physical characteristics related to the embodiments disclosed herein are not to be considered as limiting.

### DETAILED DESCRIPTION

The present invention provides non-invasive assessment of gastric function using electrophysiological analysis and digital symptom profiling of the gastric conduction system to provide actionable biomarkers that stratify patients into therapeutic groups (e.g., such as groups where gastric dysfunction is present versus absent) to provide a roadmap for personalized (e.g., patient specific) therapy. Various embodiments of the present disclosure correct and/or remove collected signals that have been corrupted by noise due to movement, adjacent anatomy signals, or the like. Failure to automatically correct noise is believed to be a large contributor to the failure of conventional systems including EGG systems. Gastric electrical signals are about 100× weaker than cardiac signals or the like and are more susceptible to noise due to anatomical location relative to other organs. Noise may be caused by movement, touching the array, poor electrode connections, muscle tension, talking, etc. These factors do not have an obvious signature that would assist in artifact removal or corrections.

Gathering simultaneous noisy and ground truth clean recordings is difficult. One may operate dedicated trials where periods of artifact are known, but there is still no ground truth to evaluate correction. Other measurement modalities are also sensitive to noise and/or cannot be administered simultaneously. The length of the test also may contribute to the difficulty in reducing noise and gathering clean data signals.

Artifact removal in gastric activity data is difficult due to the difficulty of distinguishing between gastric data and artifact data. Traditional filters such as a bandpass filters are not effective as they remove all forms of broadband activity, including genuine dysrhythmic gastric activity. One or more of the normalized biometrics described herein measure the stability of the activity in the gastric frequency band and noise appears as low stability. Therefore, a bandpass filter may not effectively identify artifacts. Embodiments of the present disclosure effectively remove artifacts if the signal cannot be recovered and correct artifacts where applicable. Embodiments of the present disclosure replace traditional signal processing filters with locked neural networks.

Various embodiments of the present disclosure describe a system that takes in a single-channel signal that may be corrupted by noise, outputs a signal that has been corrected where possible, and identifies periods of the signal where the signal has been deemed to be unrecoverable (e.g., cannot be corrected). The system may use neural networks (NNs) with parameters that are learned from a collection of existing patient data.

The data generation methodology as disclosed herein designate signal segments as “clean”, and these segments are selected to exhibit high fidelity and to accurately represent the underlying physiological phenomena. As these clean segments function as the reference, or “ground truth,” for the training of the neural network, a conservative selection protocol is employed to ensure that such segments are substantially free from spurious artifacts and reliably reflect authentic gastric rhythmic activity. The Wiener filter (WF) is utilized to extract a representative noise signature for use in the network's learning process. This methodology is based on the recognition that clean gastric signals are characterized by relatively well-defined, though variable, frequency and amplitude parameters, whereas actual artifacts encountered in practice exhibit substantially broader spectral and amplitude variability. Thus, even when the noise estimation by the WF is not ideal, it is sufficient if it encompasses salient characteristics of real-world noise. This enables the neural network to learn and correct for these noise features, thereby enhancing the fidelity of the resultant gastric signal.

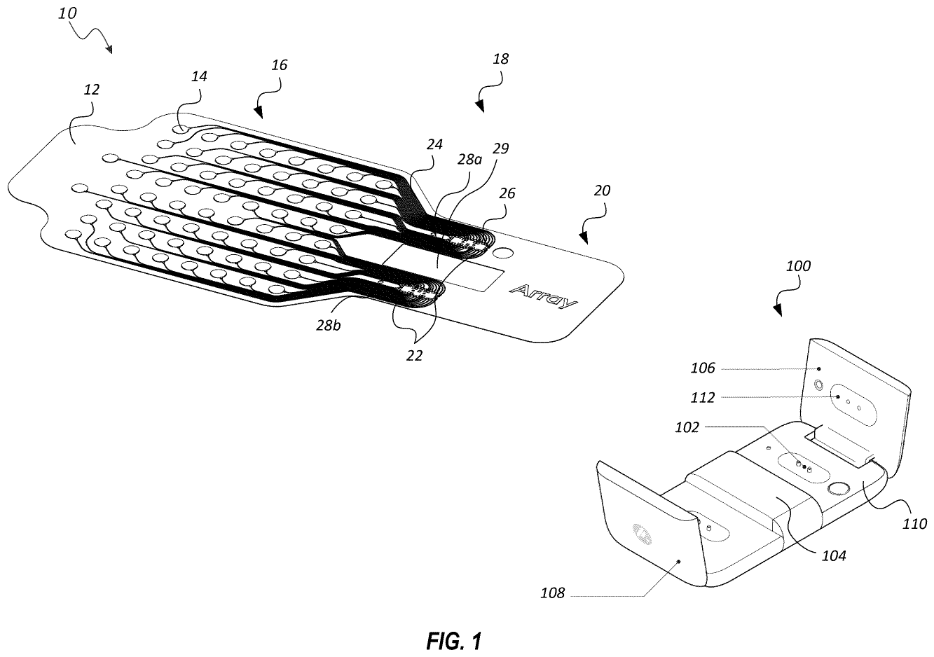

FIG. 1 is a perspective view of a flexible electrode patch. A flexible electrode patch 10 includes a flexible substrate 12 and a plurality of electrodes 14 disposed thereon. The flexible substrate 12 may include a sensing region 16, a connector region 18, and a tail region 20. In various embodiments and as shown in FIG. 1, the plurality of electrodes 14 is disposed on the sensing region 16 of the flexible substrate 12.

In various embodiments, the flexible electrode patch 10 is larger than conventional ECG patches or the like. For example, ECGs are typically recorded using a maximum of 10 electrodes that are individually applied to the patient. In another example, ECGs may be recorded via a wearable monitor that typically consists of a maximum of 4 electrodes. In contrast, the flexible electrode patch 10 of the present disclosure is relatively large to ensure that the gastric and/or colonic regions are fully covered by the plurality of electrodes 14 (e.g., including, but not limited to 64 electrodes). Furthermore, the stomach and other organs within the abdomen have variable sizes and configurations between patients and a relatively larger flexible electrode patch 10 may be used for with a wide range of patient sizes. According to at least some embodiments, the area of the sensing region 16 of the flexible electrode patch 10 is a 225 cm² (e.g., 15 cm by 15 cm). Additionally, gastric or colonic signals are relatively weak compared to signals of the heart that are measure by ECGs. Conventional ECGs would not be capable of reliably and accurately gathering the signal data compared to the flexible electrode patch 10 as described herein.

As shown in the exemplary embodiment of FIG. 1, there are total of 66 electrodes out of which 64 electrodes are arranged in an array of 8 rows and 8 columns and the remaining two electrodes are the ground and reference electrodes. In use, electrical potentials may be measured as the difference between each of the 64 electrodes and the reference electrode. The ground electrode may be the “driven right leg” or “bias” electrode. The purpose of the ground electrode in some embodiments is to keep voltage level of the subject's body within an acceptable range and to minimize any common-mode in the subject's body (e.g., 50/60 Hz power-line noise). The driven right leg may act as a source or sink. However, the flexible electrode patch 10 may comprise more than 66 electrodes or less than 66 electrodes. The ground and reference electrodes may be different than what is shown in FIG. 1. In an embodiment, the patch may comprise less than, greater than, or equal to 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275 or 300 electrodes any value or range of values therebetween in 1 increments (e.g., 33, 94, 44 to 192, etc.). According to at least some embodiments, each of the 64 electrodes (e.g., any electrodes other than the ground electrode and reference electrodes) may be equally spaced from one another. The plurality of electrodes 14 may be arranged in parallel lines (e.g., other than the ground electrode and reference electrode) or including the ground electrode and reference electrode.

According to some embodiments, for example, for a flexible electrode patch 10 having 64 electrodes as shown in FIG. 1, each electrode of the plurality of electrodes 14 may have a diameter between 10 mm and 13 mm, inclusive. Furthermore, the spacing between each of the plurality of electrodes 14 (having any number of electrodes) may be a center to center spacing between 18 mm to 22 mm, inclusive. In some embodiments, for example, for a flexible electrode patch 10 having 32 electrodes, each electrode of the plurality of electrodes 14 may have a diameter between 10 mm and 25 mm, inclusive, and a center to center spacing between 18 mm to 45 mm, inclusive.

According to various embodiments, one or more of the plurality of electrodes 14 may be deactivated during use of the flexible electrode patch 10. For example, 8 to 10 electrodes (in addition to or including the ground electrode and reference electrode) may be used for mapping in response to a determination that the 8 to 10 electrodes receive the strongest signals or the like. In various embodiments, any number of electrodes may be activated or deactivated. For example, any number of electrodes (e.g., up to and including the total number of electrodes) may be deactivated for various reasons such for saving power consumption, extending battery life, etc.

According to at least some embodiments, the flexible substrate 12 (and/or any other layers to be described herein) may be pre-formed in a convex shape such that the sensing region 16 is the first portion to contact the skin of the patient, thereby ensuring full contact between at least some of the plurality of electrodes 14 and the skin of the patient.

The flexible electrode patch 10 may further include a connector assembly 22 disposed at least partially on the connector region 18 of the flexible substrate 12. For example, the connector assembly 22 may originate at the connector region 18 and extend into the sensing region 16 and/or the tail region 20. In various embodiments, the cutout 29 is positioned between at least two connector assemblies or between at least two portions of the connector assembly 22, as shown at least in FIG. 1. A split connector assembly reduces the mating force required for the data acquisition device 100 to couple with the connector assembly 22, thereby providing a more reliable (e.g., less likely to be damaged) connection. Furthermore, the split connector assembly increases the flexibility of the flexible electrode patch 10. The combination of the cutout 29 and the split connector assembly creates a free-floating portion of the flexible electrode patch 10 that improves the conformability of the flexible electrode patch 10 to the skin of the patient, thereby increasing the reliability of the plurality of electrodes 14. The split connector assembly further simplifies manufacturing of the flexible electrode patch 10 and enables manufacturing of the relatively small plurality of electrically conductive tracks 24 of the connector assembly 22.

According to various embodiments, the flexible electrode patch 10 further includes a plurality of electrically conductive tracks 24 disposed on the flexible substrate for electrically coupling each of the plurality of electrodes 14 in the sensing region 16 and a plurality of electrically conductive contact pads 26 disposed on the connector region 18. The plurality of electrically conductive tracks 24 may be disposed on the flexible substrate 12 and run between the each of the plurality of electrodes 14 and each of the plurality of electrically conductive contact pads 26. In various embodiments, the number of the plurality of electrically conductive tracks 24 is the same as the number of the plurality of electrodes 14. Similarly, the number of the plurality of electrically conductive contact pads 26 may be the same as the number of the plurality of electrodes 14.

According to some embodiments, the plurality of electrodes 14 and/or the plurality of electrically conductive tracks 24 are screen-printed on to the flexible substrate 12 in a manner which would be appreciated by one having ordinary skill in the art. According to various embodiments, the connector assembly 22 does not include any wires (e.g., cables or the like) coupled to the flexible electrode patch 10. The omission of wires not only improves the wearability of the flexible electrode patch 10 but also simplifies manufacturing and use as wires are often difficult to make and maintain (e.g., the wires must be cleaned between each patient, etc.)

According to various embodiments, the plurality of electrodes 14 of the flexible electrode patch 10 contact the skin of a patient for use as electrophysiological sensors. Signals from the plurality of electrodes 14 may be read by connection of appropriate electronic hardware in electrical communication with the plurality of electrically conductive contact pads 26. In exemplary embodiments, the flexible electrode patch 10 is configured for use in monitoring gastro-intestinal electrical activity and/or colonic electrical of a patient, in part by an appropriate spatial arrangement of the plurality of electrodes 14 in an array, such as according to the sensor array and various embodiments as is described in WO 2021/130683, which is herein incorporated in its entirety and for all purposes.

In at least some embodiments, the connector region 18 of the flexible substrate 12 includes one or more alignment holes 28 for aligning and electrically connecting a data acquisition device 100 to the plurality of electrically conductive contact pads 26 of the connector region 18 during use. The one or more alignment holes 28 may correspond to projections 102 of the data acquisition device 100 for ensuring that the data acquisition device 100 is properly connected to the flexible electrode patch 10. In exemplary embodiments, one or more alignment holes 28 are offset for further emphasizing the correct orientation of a data acquisition device 100 relative to the flexible electrode patch 10. For example, alignment hole 28a and alignment hole 28b may be offset from each other along an axis perpendicular to a longitudinal axis of the flexible electrode patch 10, to be described in further detail below.

According to some embodiments, the data acquisition device 100 may include a first clamping member 106 and a second clamping member 108 that are configured to move between an open position as shown in FIG. 1 and a closed position having the first clamping member 106 and the second clamping member 108 adjacent to surface 110. As shown, in the open position the first clamping member 106 and the second clamping member 108 are both configured to pivotally move away from the surface 110 and reveal the surface 110 and the connector region 18 of the flexible electrode patch 10. Similarly, in the closed position the first clamping member 106 and the second clamping member 108 are both configured to move pivotally towards the surface 110 and conceal the surface 110 and the connector region 18 of the flexible electrode patch 10. The first clamping member 106 and/or the second clamping member 108 may include at least one connector 112 that is configured to be physically and operatively connected with the flexible electrode patch 10 receiving the electrical signals from plurality of electrodes 14 of the flexible electrode patch 10 to allow monitoring the electrical activity generated by the gastric or colonic activity of the patient. Therefore, no cable is required to connection between the connector assembly 22 and the flexible electrode patch 10.

In at least some embodiments, the data acquisition device 100 is free floating at the connector region 18 of the flexible substrate 12. For example, there is no adhesive coupling the flexible electrode patch 10 to the data acquisition device 100 and the flexible electrode patch 10 may conform freely to the patient's skin even with the data acquisition device 100 coupled to the flexible electrode patch 10.

In various embodiments, the flexible electrode patch 10 may include a cutout 29 for a display 104 of the data acquisition device 100 to protrude through when the data acquisition device 100 is coupled to the flexible electrode patch 10. The display 104 may include information associated with the status of the flexible electrode patch 10 and/or the data acquisition device 100 including power status, charging status, a mapping mode, etc.

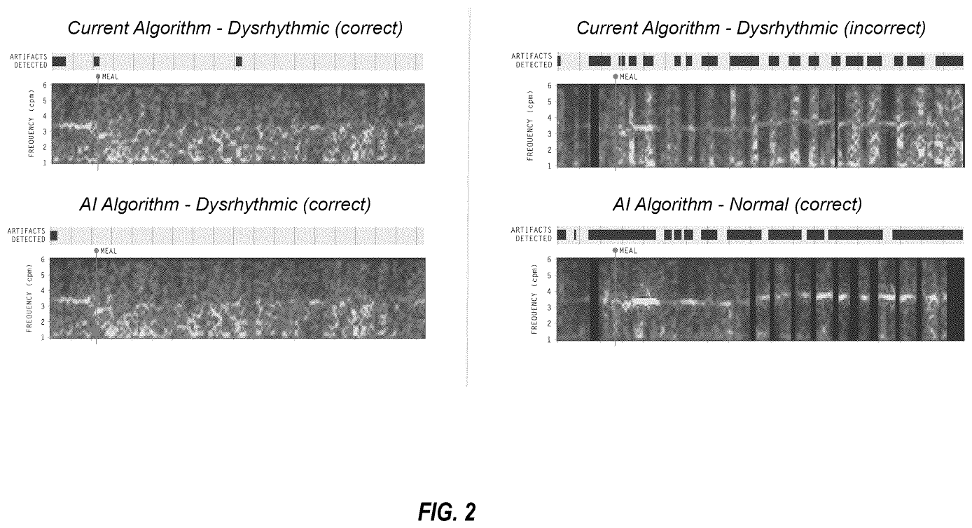

FIG. 2 is an exemplary comparison of a visual representation of electrical signals before and after noise correction. According to some embodiments, a neural-network filter (NNF) includes two primary components—a “correction” NN and a “removal” or “uncertainty” NN. The correction and removal NNs run in parallel to perform the tasks of correcting and removing periods of the data that are corrupted by noise, respectively. The training process for the correction network involves giving the network a collection of signals that have had noise added to them; the network is then evaluated on its ability to reproduce the signal with the noisy sections corrected, and it is iteratively improved based on its performance. The removal network is also trained by taking in a signal with noise added; the network is then evaluated on its ability to predict how well the correction network can estimate the noise-free clean signal. After the networks have been trained, the correction network is used to produce estimated clean signals, and the removal network is used to identify portions of the signal that could not reliably be recovered (which are subsequently removed).

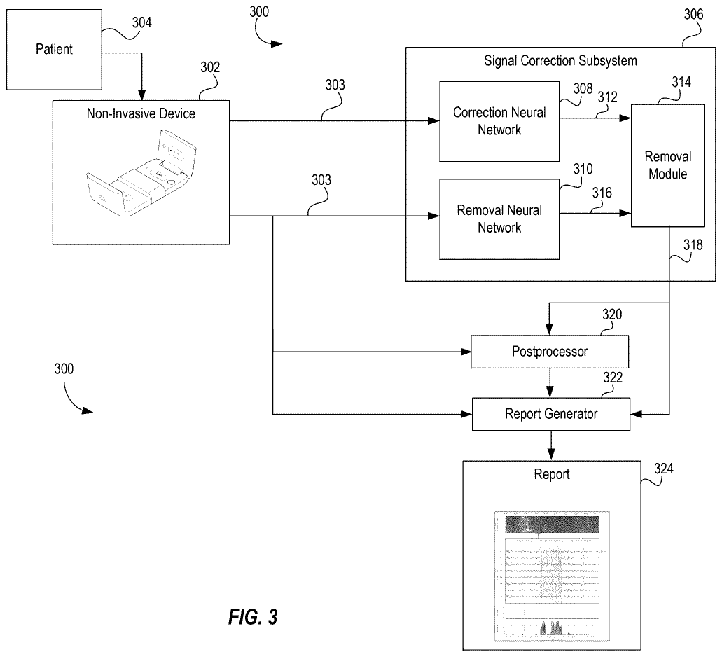

FIG. 3 is a schematic of a signal correction system. The signal correction system 300 may include various sub-systems as described herein. The system 300 may include more or less sub-systems than those explicitly described herein and the system 300 may be arranged in alternative configurations. In various embodiments, a non-invasive device 302, such as the electrode array patch and associated connector device described in detail with respect to FIG. 1, may be used to gather gastric activity data, or any other bodily activity data, of a patient 304. In particular, the non-invasive device 302 gathers electrical signals associated with gastric activity of a patient 304 over a predetermined time period.

The signal correction subsystem 306 receives the electrical signals as patient data. According to various embodiments, the patient data is received at a correction neural network 308 and a removal neural network 310 within the signal correction subsystem 306. The correction neural network 308 and the removal neural network 310 may be convolutional neural networks. The correction neural network 308 may output an estimate of the clean data signals and the removal neural network 310 may output a confidence score associated with the signals. The removal neural network 310 may use the same electrical signals received at the correction neural network 308 and specifies data segments to be removed based at least in part on the confidence score, to be described in further detail below.

For each received signal, the signal is provided and an input signal 303 to the correction neural network 308 and the removal neural network 310. The correction neural network 308 outputs an intermediate corrected signal 312 to the removal module 314. The intermediate corrected signal 312 may be generated based at least in part on the input signal 303 and each segment of the input signal 303 identified by the correction neural network 308 to contain noisy data may be replaced with a corrected segment generated by correction neural network 308.

The removal neural network 310 outputs a confidence score 316 for each segment of input signal 303 identified by the removal neural network 310 to contain noisy data. The confidence score 316 may represent an estimated difference between a true noise-free signal 303 and a predicted corrected signal. In response to determining that the confidence score 316 associated with the corrected segment is below a preconfigured threshold value, the removal module 314 replaces the corrected segment with a segment representing deleted data to generate a final corrected signal 318.

According to various embodiments, the final corrected signal 318 is output to a postprocessor 320 and/or a report generator 322. The postprocessor 320 and/or the report generator 322 may also receive the input signal 303. The postprocessor 320 performs various postprocessing operations to be described in further detail below. The report generator 322 generates a final report 324 based on the final corrected signal 318. The report 324 may identify a gastrointestinal phenotype based at least in part on the final corrected signal 318, according to at least some embodiments.

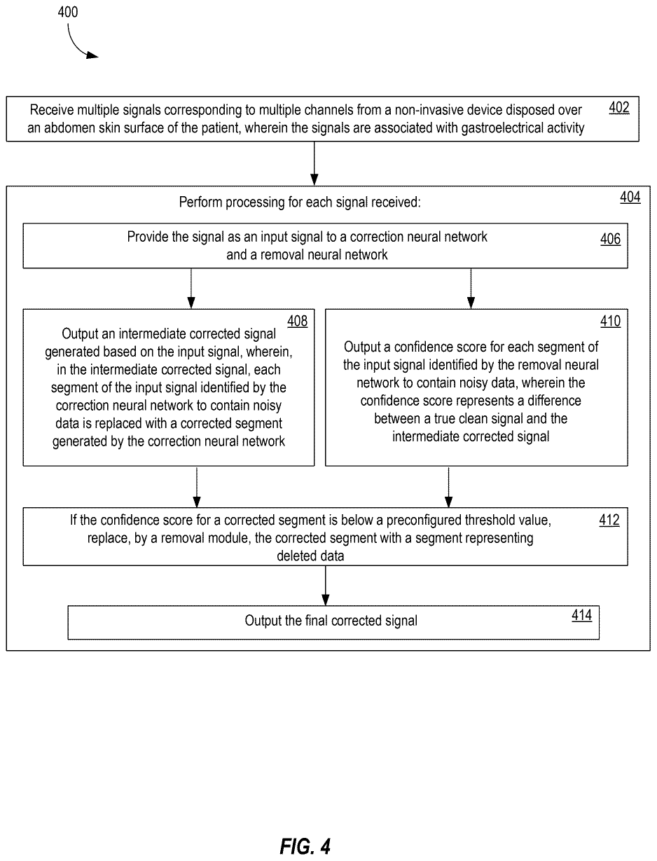

FIG. 4 is a flowchart of a method of processing gastric activity data. Method 400 may include various operations that may be performed in alternative configurations than that described herein. Method 400 may include more or less operations than those described herein. Method 400 may include operation 402. Operation 402 includes receiving electrical signals associated with gastric activity of a patient over a predetermined time period. The predetermined period of time may be intervals of 30 mins, 1 hour, 2 hours, etc. In exemplary embodiments, the predetermined period of time is at least 4 hours. The electrical signals may be measured with an electrode array patch disposed over an abdomen skin surface of the patient. The electrical signals may be indicative of and/or associated with gastric activity, colonic activity, or any combination thereof. In various embodiments, the electrical signals are indicative of a gastrointestinal phenotype for use in prescribing treatment for a patient.

According to at least some embodiments, a channel of data is received for each electrode of the electrode array patch applied to a patient. For example, for an electrode array patch having 64 electrodes, 64 channels of data may be received. In at least some embodiments, method 400 may including selecting a plurality of channels for performing various operations. For example, channels having the “cleanest” data may be selected and channels having relatively more noise (e.g., due to touching or poor connection) may be removed from the analysis and processing operations.

Method 400 may include operation 404 including several operations that are performed to process each signal received in operation 402. Operation 406 includes providing the signal as an input signal to a correction neural network and a removal neural network. Operation 406 may be performed in sequence or simultaneously according to various embodiments.

Operation 408 includes outputting, by the correction neural network, an intermediate corrected signal generated based on the input signal. In the intermediate corrected signal, each segment of the input signal identified by the correction neural network to contain noisy data is replaced with a corrected segment generated by the correction neural network. In some embodiments, an initial report may be generated based on the intermediate corrected signal. The initial report may include a gastrointestinal phenotype based at least in part on the intermediate corrected signal.

Operation 410 includes outputting a confidence score for each segment of the input signal identified by the removal neural network to contain noisy data. The confidence score may represent an estimated difference between the true noise-free signal and a predicted corrected signal as quantified by an estimate of the variance that maximizes the Gaussian negative log likelihood of the true noise-free point given an estimated mean (corrected signal).

Operation 412 includes determining whether each confidence score associated with a corrected segment is below a preconfigured threshold value. If the confidence score for a corrected segment is below a preconfigured threshold value, a removal module may replace the corrected segment with a segment representing deleted data.

Operation 414 includes outputting the final corrected signal. Operation 414 may further include generating a final report based on the final corrected signal. The final report may include a visual representation of the final corrected signal. The visual representation may identify data segments replaced by corrected segments and/or data segments replaced by segments representing deleted data. For example, the corrected and/or removed segments may be identified by color, label, text, etc., or combination thereof, in a visual representation of the final corrected signal. The report may identify the gastrointestinal phenotype based at least in part on the final corrected signal. The gastrointestinal phenotype may be a dysrhythmic, high frequency, low meal response, sensorimotor, continuous, or delayed onset phenotype.

According to various embodiments, method 400 may further include determining one or more normalized biometrics over at least a predetermined time period from the final corrected signal. The one or more normalized biometrics may include at least one of a principal gastric frequency (PGF), a body mass index (BMI)-adjusted amplitude, Gastric Alimetry Rhythm Index (GA-RI), fed:fasted amplitude ratio (ff-AR), and a meal response ratio.

In some embodiments, method 400 may further include outputting a recommendation based at least in part on the gastrointestinal phenotype associated with the patient. For example, various medications, diets, therapies, or any combination thereof, may be included in the recommendation, to be further verified by the health care provider receiving the final report.

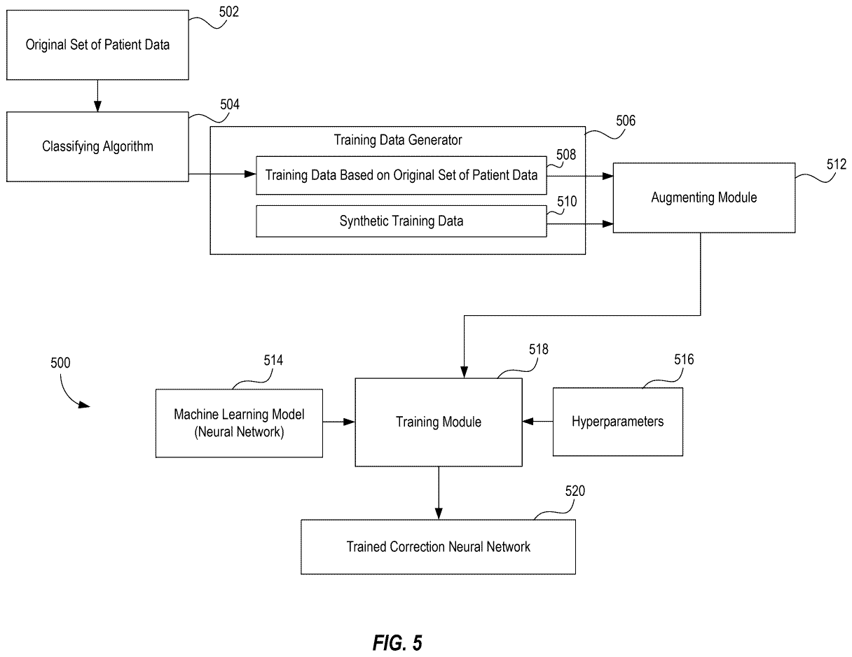

FIG. 5 is a schematic of a signal correction sub-system for training a correction neural network. Various operations of method 400, or other methods described herein, may be performed by a correction neural network. The correction neural network may be trained according to embodiments described herein. The signal correction sub-system 500 may include various sub-systems as described herein. The sub-system 500 may include more or less sub-systems than those explicitly described herein and the sub-system 500 may be arranged in alternative configurations. In various embodiments, an original set of patient data 502 is provided. The original set of patient data 502 may include historical patient data, patient data received in real-time, or any combination thereof. A classifying algorithm 504 may perform various preprocessing operations on the original set of patient data 502 before the original set of patient data 502 is input into a training data generator 506. Preprocessing may include various existing algorithms known in the art including, for example, a noise filter, a bandpass filter, an impedance check, a spatial smoother, a channel ranker, etc.

The training data generator 506 generates training data based on the original set of patient data 508. The training data generator 506 may further include synthetic training data 510. The training data based on the original set of patient data 508 and/or the synthetic training data 510 may be input into an augmenting module 512.

According to various embodiments, the output of the augmenting module 512, a machine learning model 514 (e.g., a neural network), hyperparameters 516, etc., may be input into a training module 518. The training module 518 outputs a trained correction neural network 520.

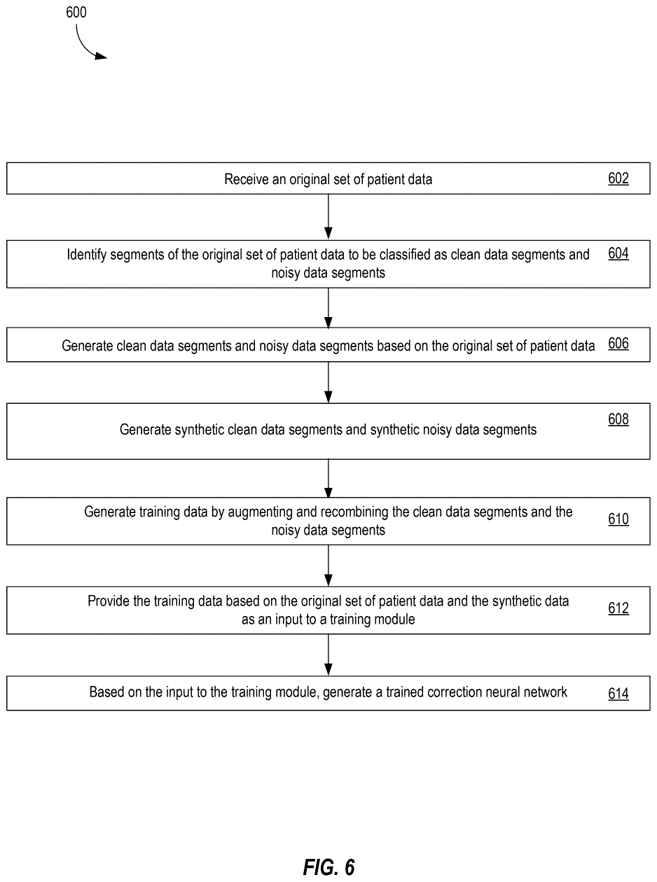

FIG. 6 is a flowchart of a method of training a correction neural network. Method 600 may include various operations that may be performed in alternative configurations than that described herein. Method 600 may include more or less operations than those described herein. Method 600 may include operation 602. Operation 602 includes receiving an original set of patient data. The original set of patient data may include one or more channel of electrical signal data wherein each channel is associated with an electrode of the electrode array patch, as described in detail above.

Operation 604 includes identifying segments of the original set of patient data to be classified as clean data segments and noisy data segments. Clean data segments may be any length or a combination of lengths. According to some embodiments, clean data segments may be data segments characterized as having a noise level at or below a predetermined threshold. Noisy data segments may similarly be any length or combination of lengths and may be data segments characterized as having a noise level greater than or equal to a predetermined threshold. Noisy data segments generally include more artifacts to be corrected or removed as compared to clean data segments.

Operation 606 includes generating clean data segments and noisy data segments based on the original set of patient data. In various embodiments, operation 606 includes generating a collection of segments that are known to be clean (e.g., would constitute a suitable output of the model) and corresponding segments with areas corrupted by noise.

Operation 608 includes generating synthetic clean data segments and synthetic noisy data segments. Generating the synthetic data may include synthesizing clean data segments and noisy data segments based on the original set of patient data.

Operation 610 includes generating training data by augmenting and recombining clean and noisy data segments. The clean and noisy data segments may each be generated synthetically and/or determined based on the original set of patient data. According to various embodiments, at operation 610 four types of data segments may be provided including clean patient data segments, noisy patient data segments, clean synthetic data segments, and noisy synthetic data segments. The clean data (e.g., the clean patient data and/or the clean synthetic data) represents data that would be a suitable output from the correction network. The noisy data (e.g., the noisy patient data and/or the noisy synthetic data) represents data that can be added to the clean data (via the augmenting module) to be input to the neural networks for training purposes. The patient data (clean or noisy) is data from actual testing using the electrode array patch described herein and the patient data has been classified as clean or noisy. The synthetic data (clean or noisy) is data that is synthesized completely independently from any real data (e.g., via generating signals at a random frequency and adding random noise). The augmenting module combines clean data (patient or synthetic) with noisy data (patient or synthetic).

Operation 612 may include providing the training data based on the original set of patient data and the synthetic data as an input to a training module. Operation 612 may further include training the training module using the training data based on the original set of patient data and the synthetic data.

Operation 614 may further include, based on the input to the training module, generating a trained correction neural network.

In some embodiments, the terms training, tuning, and testing to refer to the various data sets (the term “validation” is often used in place of “tuning” in machine learning contexts and used in place of “testing” in regulatory contexts). The splitting of these datasets occurs at the subject level, i.e. data from a single test will only be used in at most one of these datasets. According to some embodiments, three data sets may be used including: training data, tuning data, and test data. Training data may be used to directly update the weights of the NNs through an iterative loss-minimization process. Tuning data may be used to estimate the performance of the NNs on data that has not been used directly to update network weights for the purpose of identifying optimal hyperparameters, model architectures, training stopping times, etc. Test data may be used to assess whether or not the final model (i.e. the single model that performs best on the tuning data) is suitable for deployment. The test data is employed in a formal test protocol that yields a pass/fail outcome. In addition to excluding all data from the training/tuning sets, the test set may also exclude all data used in previous executions of the test protocol. In other words, a given subject can only be used in the testing protocol at most once, otherwise they will have effectively been used as tuning data. Testing data may also have further requirements with respect to demographic representation, etc.

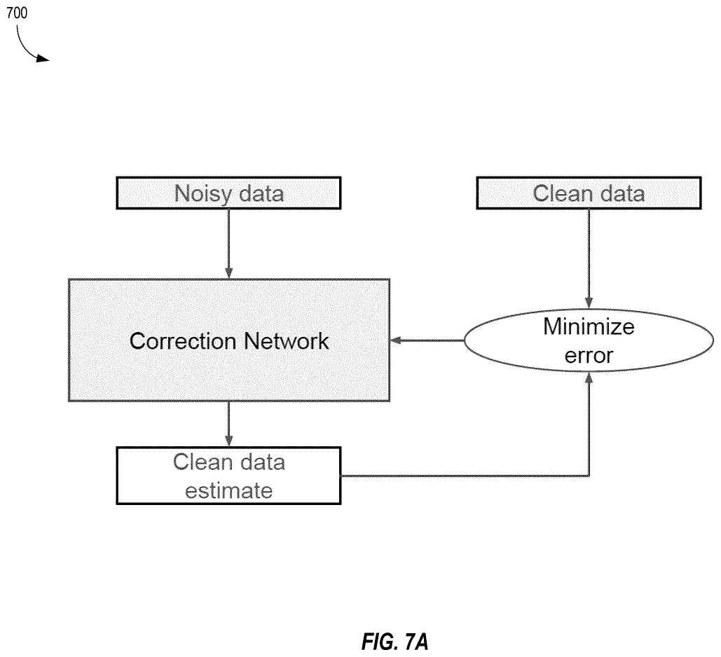

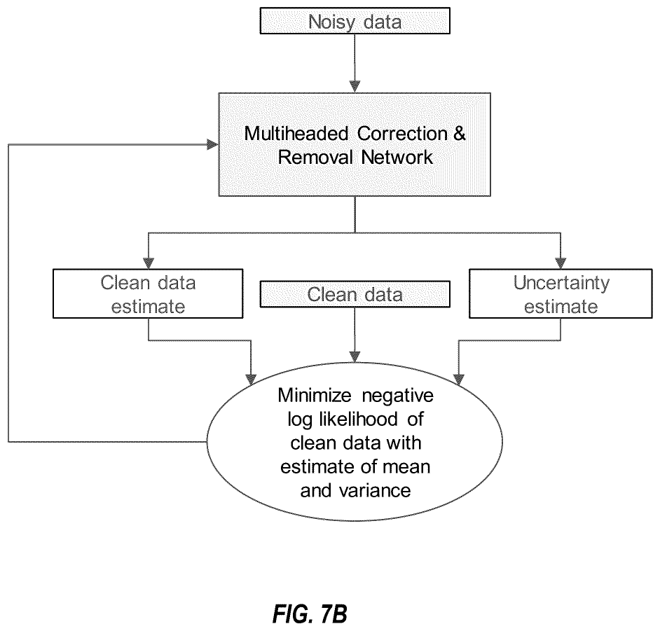

FIG. 7A is a schematic of a correction neural network. The correction neural network 700 may be trained to transform noisy data into data matching the true clean data. The correction neural network 700 may be architected as a convolutional neural network with non-linear activation functions, except for the output activation, which is linear so that the network can estimate both positive and negative values. According to various embodiments, the correction neural network 700 utilizes skipped or extra connections.

According to various embodiments, the correction neural network 700 utilizes variable dilation. Dilation refers to the spacing kernel weights that are being convolved with the layer inputs. Increased dilation has the effect of increasing the “receptive field” (i.e. the scale of the features which may be recognized by a given filter/layer) without having to increase the number of parameters (as happens with increased kernel size).

According to various embodiments, the correction network uses a residual U-Net architecture with increased dilations in layers closer to the middle of the network and skipped connections joining layers with equivalent dilations at opposing ends of the network.

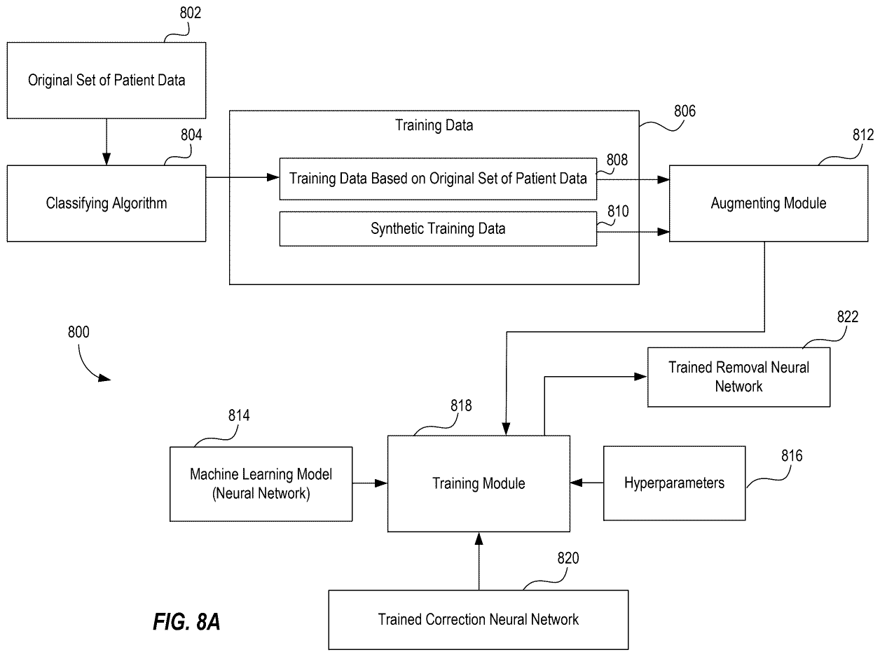

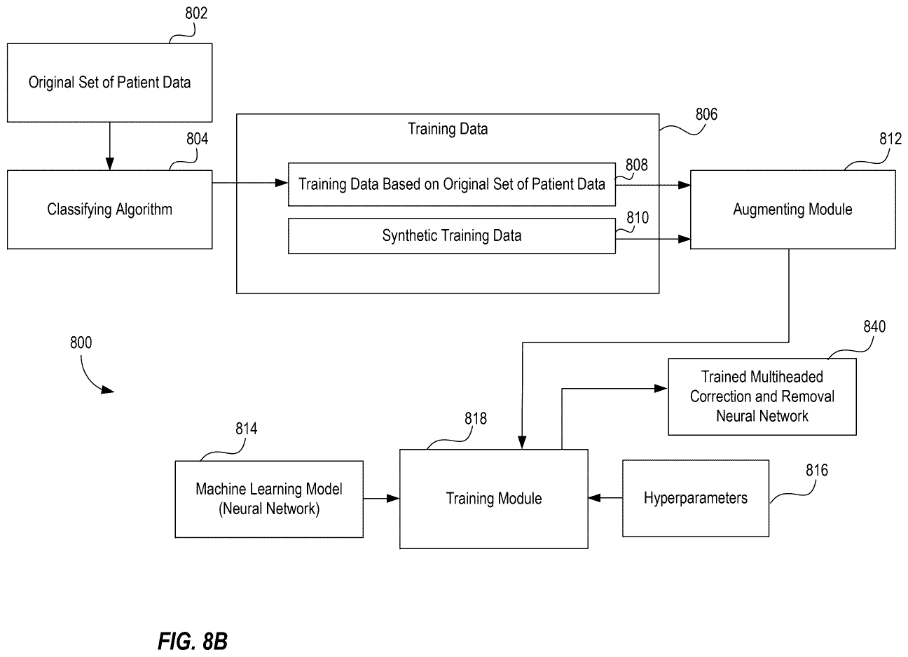

In some embodiments, the removal neural network may be architected as a convolutional neural network with non-linear activation functions. The output activation may be any non-negative function to enable estimation of uncertainty values, including Rectified Linear Unit (ReLU, and its variants), Softplus, Softmax, Soft-Absolute. The neural network architecture may be the same or different from that of the correction network. In some embodiments, the correction and neural networks may share weights and have multiple “heads” used to estimate the clean signal and corresponding uncertainty simultaneously, i.e., be comprised of a single network with multiple outputs as shown in FIG. 7B and in further detail with respect to FIG. 8B including a trained multiheaded correction and removal neural network 840.

The correction neural network 700 may be trained to output an estimate of the clean signal when receiving the combined signal (clean+noise) as input. A standard NN training pipeline where stochastic gradient descent may be used to minimize a loss function. After each training epoch (iteration through complete training set), the average loss may be calculated on the tuning set without making any changes to the NN weights. The loss function used to train the correction network characterizes the accuracy with which the true clean signal is estimated, such as the mean squared error loss, L1 loss, Huber loss, Log-Cosh loss, or spectral loss functions. Training may also be performed using a loss function that normalizes the error relative to the amplitude of the ground-truth signal and reduces the penalty for errors at time points with high-amplitude noise, such that there is higher importance placed on accuracy for cleaner portions of the signal, given the inherent difficulty of perfectly reconstructing signals in the presence of severe artifacts. In various embodiments, the loss function evaluates the error between an estimated and a true signal with normalization used such that losses errors are measured proportionally to the clean signal magnitude and/or with pointwise scaling such that greater loss occurs for errors at time points where there was less corruption from noise. For example, the loss function utilized in training the correction neural network may include an adaptive scaling factor for each time point, the factor being inversely related to an estimate of noise present in the true signal at that time point.

According to some embodiments, the correction network was trained using a scaled L1 loss function. Standard L1 loss calculates the mean absolute error between the prediction and the ground truth. The modified loss function as disclosed herein introduces two scaling factors. First, it normalizes the error relative to the amplitude of the ground-truth signal, ensuring that a 1 μV error on a 10 μV signal is weighted similarly to a 10 μV error on a 100 μV signal, thereby accounting for the significant inter-individual variability in amplitude. The loss function as disclosed herein further reduces the penalty for errors at time points with high-amplitude noise. Accordingly, the presently disclosed loss function prioritizes accuracy on cleaner portions of the signal, acknowledging the inherent difficulty of perfectly reconstructing signals from severe artifacts.

In some embodiments, the loss L for a single sample comprised of noisy input x, a clean output y, and an estimated clean signal yₑₛₜ, is the mean of the element-wise L1 loss across all time steps T, scaled by a factor S(t) that is inversely related to the local noise magnitude, and normalized by the L2 norm of the true clean output:

$$L ( y , {y}_{est} , x ) = \frac{1}{T \cdot {‖ y ‖}_{2}} {\sum }_{t = 1}^{T} S ( i ) \cdot \vert {y}_{est} ( t ) - y ( t ) \vert$$

The scaling factor is calculated using the normalized instantaneous noise magnitude and two hyperparameters that determine the weighting assigned to the noise magnitude when scaling (Nₘₐₓ) and the maximum allowable reduction (Rₘₐₓ):

$$S ( t ) = 1 - ( \frac{1}{{N}_{max}} \cdot \frac{\vert x ( t ) - y ( t ) \vert}{{‖ y ‖}_{2}} , {R}_{max} )$$

In this study, Nₘₐₓ=2 and Rₘₐₓ=0.9. Nₘₐₓ may be interpreted as dictating that there should be zero loss (i.e., a reduction of 1, such that S(t)=0) when the noise magnitude is twice the standard deviation of the true clean signal. However, setting a max reduction of 0.9 ensures that all time points are factored into the loss, with estimation errors at time points for which there is no noise at the input having 10× loss as compared to equivalent errors at time points with large noise magnitude.

During training, this loss is calculated for every sample in the batch. The final loss for the batch is the mean of all these individual sample losses. This batch loss is then used to update the network's weights, and this process is repeated for all batches in the training dataset.

The removal network was trained using the Gaussian Negative Log Likelihood (GNLL) loss for uncertainty estimation. The GNLL trains the network to predict the variance associated with the correction network's estimated clean signal. The loss is minimized when the uncertainty network outputs a high variance for predictions where the correction network has a relatively large error, and a low variance for predictions where the error is relatively small.

Training may be performed in sequence on the correction and removal networks, according to some embodiments. For example, a correction network may be trained to convergence. The correction network may be used to produce clean signal estimates for all of the data used for training the uncertainty network. These estimates, along with the ground-truth clean signals, may be used as the mean and point of evaluation in the GNLL loss, and the uncertainty network weights updated based on the loss calculated when using the pointwise outputs as the estimated variance in the GNLL loss. In other embodiments, the models may be trained in parallel using a joint GNLL, i.e. such that the correction network and removal network simultaneously estimate the mean and variance of a Gaussian distribution and are both updated according to the GNLL loss.

In an exemplary embodiment, various models may be built and trained using Pytorch. Stochastic gradient descent may be performed using the Adam optimizer with a batch size of 32. 7-minute segments may be used for training. For the correction network, the augmented datasets may include 1,500,000 segments for training and 30,000 segments for tuning, trained with a learning rate of 1e-5. For the removal network, the augmented datasets may include 1,000,000 segments for training and 30,000 segments for tuning, trained with a learning rate of 1e-6. Synthetic data may be used only for training the correction network in this exemplary embodiment.

FIG. 8A is a schematic of a signal correction sub-system for training a removal neural network. Various operations of method 400 or method 900, or other methods described herein, may be performed by a removal neural network. The removal neural network may be trained according to embodiments described herein. The signal correction sub-system 800 may include various sub-systems as described herein. The sub-system 800 may include more or less sub-systems than those explicitly described herein and the sub-system 800 may be arranged in alternative configurations. Various embodiments the sub-system 800 may be applicable to the sub-system 800 and similar numbering indicates similar form and functions unless otherwise noted herein.

In various embodiments, an original set of patient data 802 is provided. The original set of patient data 802 may include historical patient data, patient data received in real-time, or any combination thereof. A classifying algorithm 804 may perform various preprocessing operations on the original set of patient data 802 before the original set of patient data 802 is input into a training data generator 806. Preprocessing may include various existing algorithms known in the art including, for example, a noise filter, a bandpass filter, an impedance check, a spatial smoother, a channel ranker, etc.

The training data generator 806 generates training data based on the original set of patient data 808. The training data generator 806 may further include synthetic training data 810. The training data based on the original set of patient data 808 and/or the synthetic training data 810 may be input into an augmenting module 812.

According to various embodiments, the output of the augmenting module 812, a machine learning model 814 (e.g., a neural network), hyperparameters 816, etc., may be input into a training module 818. The training module 818 outputs a trained correction neural network 820. Outputs of the trained correction neural network 820 and the training module 818 may be input to a trained removal neural network 822 according to various embodiments.

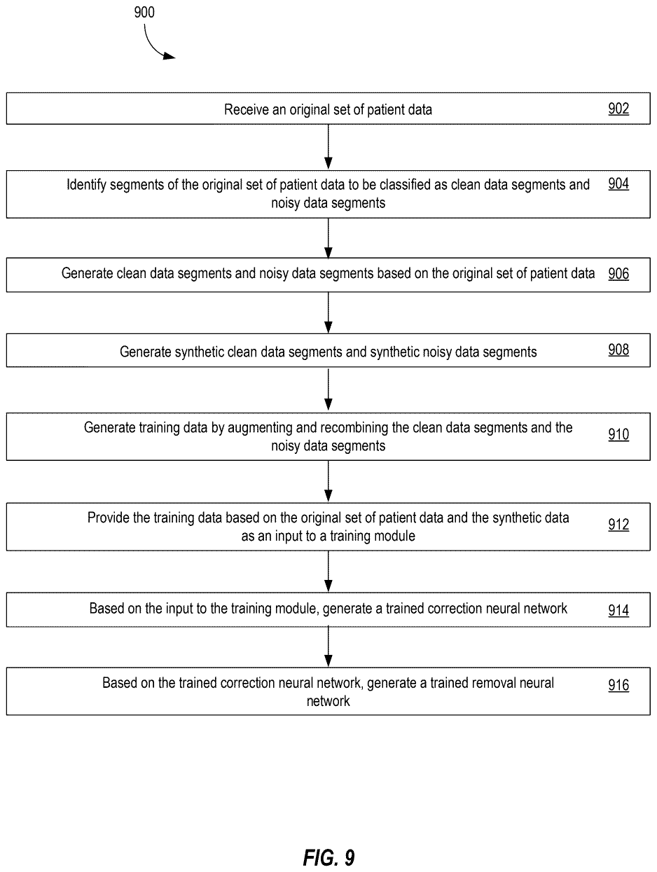

FIG. 9 is a flowchart of a method of training a removal neural network. Method 900 may include various operations that may be performed in alternative configurations than that described herein. Method 900 may include more or less operations than those described herein. Method 900 may include operation 902. Operation 902 includes receiving an original set of patient data. The original set of patient data may include one or more channel of electrical signal data wherein each channel is associated with an electrode of the electrode array patch, as described in detail above.

Operation 904 includes identifying segments of the original set of patient data to be classified as clean data segments and noisy data segments. Clean data segments may be any length or a combination of lengths. According to some embodiments, clean data segments may be data segments characterized as having a noise level at or below a predetermined threshold and a dominant frequency within a target frequency band. Noisy data segments may similarly be any length or combination of lengths and may be data segments characterized as having a noise level greater than or equal to a predetermined threshold. According to some embodiments, requirements may be placed on the duration of clean or noisy data segments.

Operation 906 includes generating clean data segments and noisy data segments based on the original set of patient data. Operation 906 may include running a version of a Wiener Filter (WF) on each channel of the available recordings to produce a label of “noisy” or “clean” for every timepoint. For samples labeled noisy, the algorithm also provided an estimate of the sample value with the noise removed. Consecutive clean samples may be stored as a clean segment. An individual clean segment may be of an arbitrary length. Consecutive noisy samples may be considered a segment during which noise occurred. To get an estimate of the noise signature, the estimated corrected signal was subtracted from the original noisy segment. According to various embodiments, the clean signals are used as target outputs from the NN-based filter. In exemplary embodiments, the NN is trained to output signals for which there is high confidence in their legitimacy as gastric rhythms.

Operation 908 includes generating synthetic clean data segments and synthetic noisy data segments. Generating the synthetic data may include synthesizing clean data segments and noisy data segments based on the original set of patient data. When training the correction NN, the real data segments extracted from the patient data may be supplemented with synthetic data. For example, three types of synthetic data generation processes may be used to generate synthetic data. First, clean signals may be generated with stable rhythms. These signals simulate cases where there is clearly measured gastric signal without large noise artifacts. Second, the generation of clean signals with scattered rhythmic activity. These signals simulate cases where the gastric activity is clearly recorded without any corruption from noise, but the signals do not have a clear stable rhythm (as may be the case for diseased stomachs). Third, the generation of noise segments.

According to some embodiments, synthetic data is only used in network training, not in tuning or testing to minimize any biasing effect from the nature of the simulated signals. Specifically, by only using real data for network tuning, this ensures that the network configuration/training epoch that is selected is optimized to recover real data segments.

According to various embodiments, the synthetically generated stable rhythms are intended to represent a broad range of possible gastric signals. The frequency and amplitude of the constructed signal are selected randomly from a broad range of possible frequencies and amplitudes that is reflective of the highly variable nature of these parameters in actual gastric signals. Additionally, both the amplitude and phase of the signal accumulate a random drift. Including a drift in phase introduces a “wobble” in the signal such that it is not a perfect sinusoid, and also produces a slight drift in frequency over time.

Since not all gastric signals display stable rhythmic activity, synthetic signals with unstable, or “scattered”, activity are also generated. The scattered activity signals are generated by combining multiple stable signals at different frequencies, with each component having an increased amount of phase drift as compared to the stable signals.

Noisy segments are constructed by multiplying random noise with a curve to simulate the effect of noise ramping up and down. The shape of the curve is determined by some randomized parameters to promote variability in the noise patterns. The duration of noise segments is highly variable, determined by a randomly generated number.

Operation 910 includes generating training data by augmenting and recombining clean and noisy data segments. The clean and noisy data segments may each be generated synthetically and/or determined based on the original set of patient data. Training and tuning datasets may be constructed by adding noise segments to clean segments to generate samples of realistic noisy data (the combined segment) for which the ground truth (corresponding clean segment) is known. The number of noise segments added to a clean segment may be variable and/or randomized to promote varying degrees of signal contamination. The clean data segments may be modified to have a different amplitude prior to combination with noise. In addition to enabling knowledge of the ground truth clean data, this data augmentation procedure allows for there to be a large number of samples for use in training, as clean segments can be reused in multiple combined segments (i.e. with different noise segments). The datasets may be generated using the same procedure for training and tuning of both the correction and removal networks, with the only difference being whether or not synthetic data is included

According to some embodiments, for the instances where only patient data is used (i.e. tuning the correction network and training/tuning the removal network), every sample is generated using real data for both the clean and noisy data segments. The function for creating a dataset requires a collection of clean and noisy segments of patient data to be provided. The datasets used for training and tuning are thus created by providing this function with segments taken from non-overlapping groups of subjects. Lastly, the dataset creation process takes a random seed (enabling control over all steps involving random number generation), which allows for the dataset to be reproducible across multiple training runs for the purpose of performance comparison.

Operation 912 may include providing the training data based on the original set of patient data and the synthetic data as an input to a training module. Operation 912 may further include training the training module using the training data based on the original set of patient data and the synthetic data.

Operation 914 may further include, based on the input to the training module, generating a training correction neural network.

According to various embodiments, based on the trained correction neural network, a trained removal neural network may be generated in operation 916.

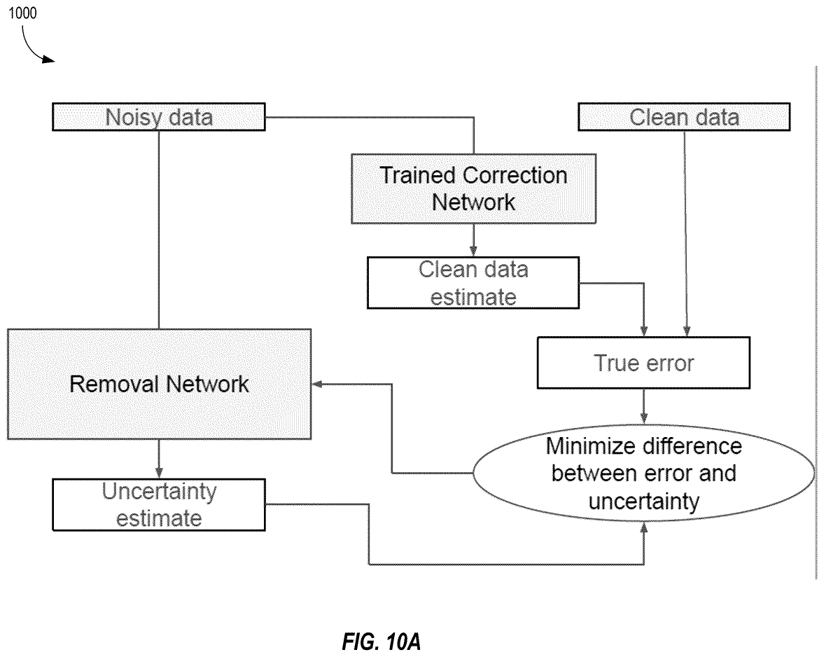

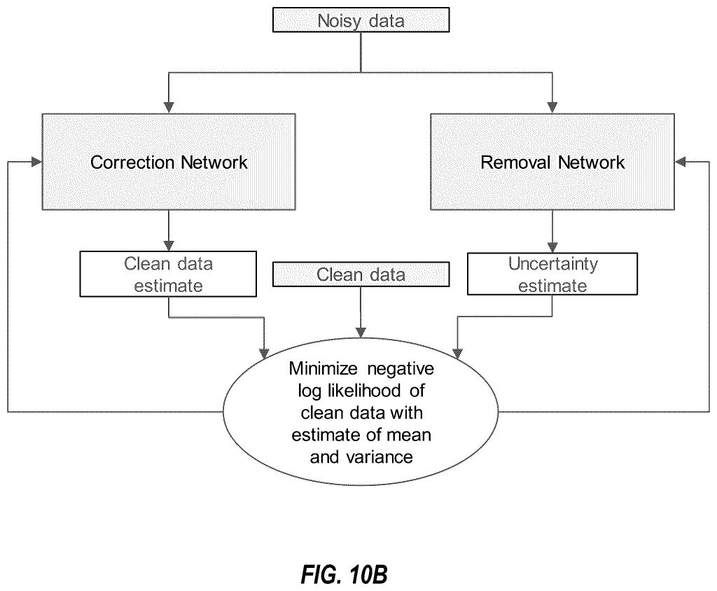

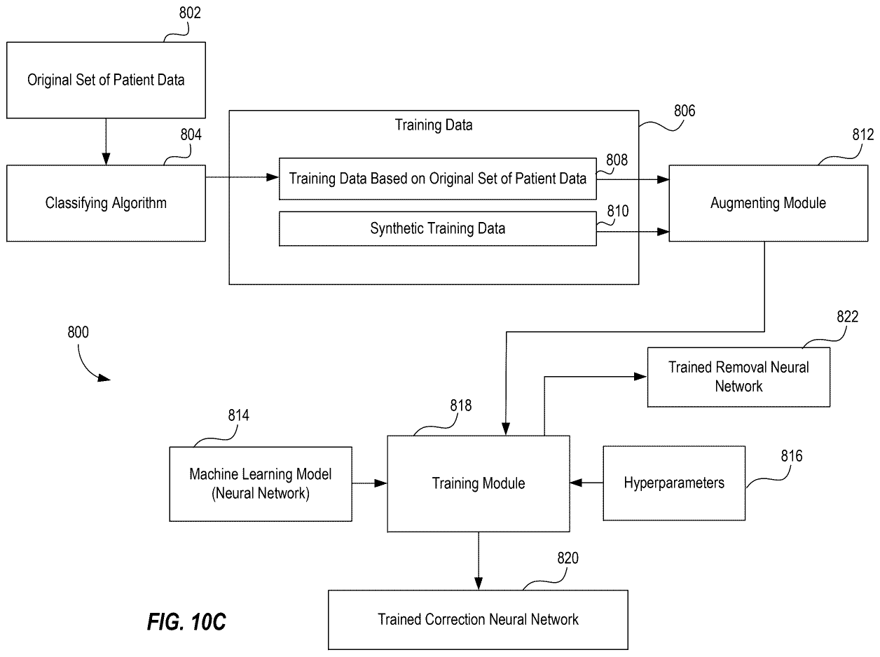

FIG. 10A is a schematic of a removal neural network. The removal neural network 1000 learns to estimate a confidence interval for the estimate produced by a trained correction network, such as the correction neural network 700. The removal neural network 1000 finds where there is significant noise (as opposed to recovering what the true signal was where there is noise). This training process may use a loss function that quantifies how well the removal network estimates the magnitude of the error of the estimated clean signal with respect to the true clean signal, such as the mean squared error loss, L1 loss, Huber loss, Log-Cosh loss, or spectral loss functions, where the loss is applied to the estimated absolute (or squared) error against the true absolute (or squared) error between the true and estimated clean signals. The loss function may be the Gaussian Negative Log Likelihood loss, calculated using an estimated variance of a Gaussian distribution centered at the true clean signal and evaluated at the estimated clean signal. The correction network remains locked for this process, such that the estimated clean signal (and true absolute errors) can be used as inputs for the removal network loss functions. In some embodiments, the correction and removal networks may be trained simultaneously, as shown in FIG. 10B and FIG. 10C, using the Gaussian Negative Log Likelihood loss to evaluate both the estimated variance and estimated clean signals and update the weights of both networks accordingly.

The noise filter module of the preprocessing pipeline involves converting 64-channel data that may contain noise into an estimate of the corrected 64-channel data and a per-channel/per-sample mask indicating where data should be removed. The NNF implementation uses the correction and removal networks in parallel to produce these two outputs from the 64-channel noisy data. To produce the estimated clean signals, the correction network may be applied to each channel independently. The architectures of both NNs are agnostic to the size of the input data, meaning that they can process arbitrary length inputs (e.g., 4.5 hours) despite having been trained on 7-minute segments. To produce the removal mask, the removal network produces a per-channel/per-sample estimate of the error relative to the local amplitude of the signal, which is compared to a prespecified threshold.

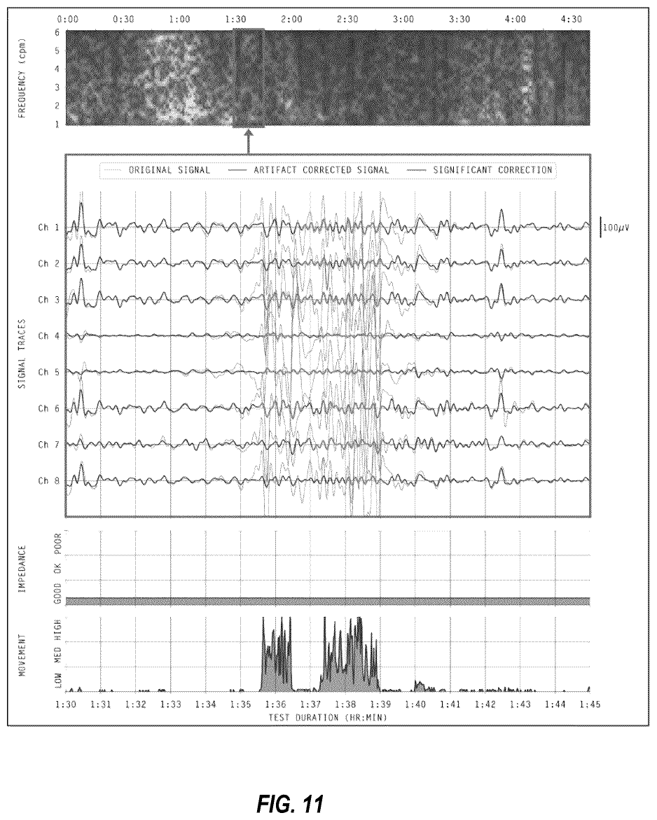

FIG. 11 is an exemplary report of gastric activity data. The report may include various visual representations of the original set of patient data and/or the patient data as modified (e.g., corrected, removed, etc.) as described herein. For example, each channel of signal data used for the analysis may be shown with various corrected or replaced data segments highlighted by a different line thickness, pattern, color, or any combination thereof.

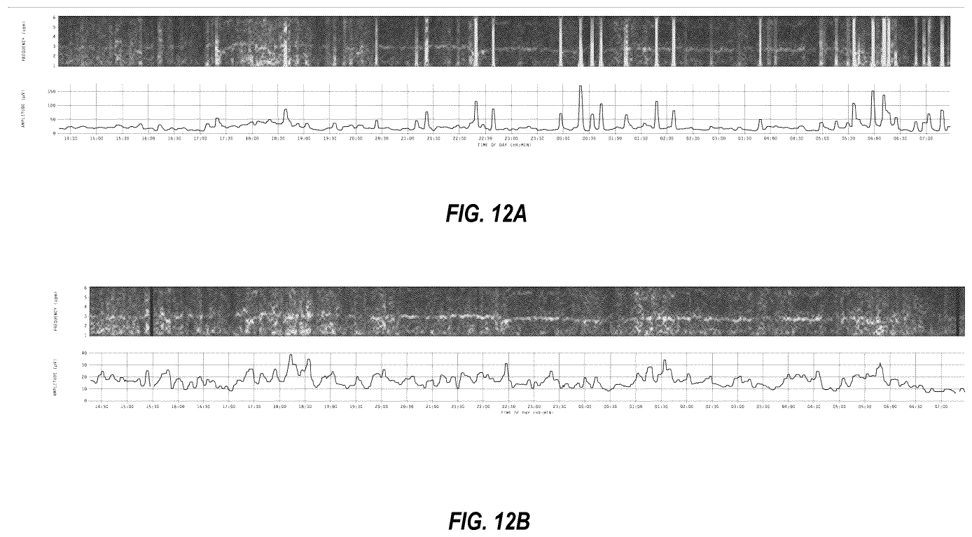

FIGS. 12A-12B illustrate an exemplary comparison of a visual representation of electrical signals before and after noise correction. In particular, FIGS. 12A-12B illustrate a ˜17 hr ambulatory recording with and without the neural network artifact removal according to embodiments of the present disclosure. FIGS. 12A-12B demonstrate the importance of the artifact removal for providing reliable and accurate ambulatory recordings.

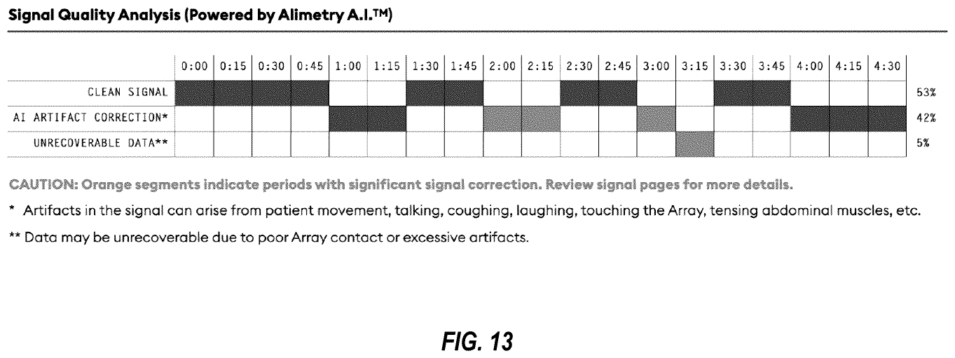

FIG. 13 is an exemplary signal quality report. In various optional embodiments, a signal quality report may be provided with an initial report and/or a final report that indicates clean data segments, corrected data segments, unrecoverable data, etc. A confidence score may be shown as associated with each segment and colors/text may be used to provide a caution or warning to a health care professional and/or patient reviewing results of the test.

### Results

The final locked NNF was evaluated on an independent test cohort of 127 patients (37,177 test segments), with no data overlap from the training or tuning sets. The NNF demonstrated statistically significant and clinically meaningful improvements over the traditional WF (Table 1). The NNF achieved a 6.39% absolute reduction in Mean Absolute Percentage Error (MAPE) compared to the WF (p<0.0001), indicating superior reconstruction accuracy. Furthermore, the NNF reduced the percentage of data removed by 1.07% (absolute), demonstrating a greater capacity to preserve data by confidently reconstructing signals that the WF would have discarded, thereby retaining more potentially valuable clinical information. The key quantitative performance metrics are summarized in Table 1.

TABLE 1
Results of the evaluation on a held out clinical cohort. Results
provided as mean (standard deviation) across patients.

| | Neural | | |
|---|---|---|---|
| | Network | Wiener | Paired |
| Performance | Filter | Filter | t-test |
| Metric | (NNF) | (WF) | p-value |
| Mean Absolute | 9.97 (30.81) | 16.36 (38.46) | <0.0001 |
| Percentage Error | | | |
| (MAPE) | | | |
| Percentage of Data | 10.22 (11.21) | 11.28 (7.28) | <0.0001 |
| Removed | | | |
| Artifact-Movement | 0.096 (0.079) | 0.124 (0.085) | <0.0001 |
| Correlation | | | |
| Coefficient | | | |

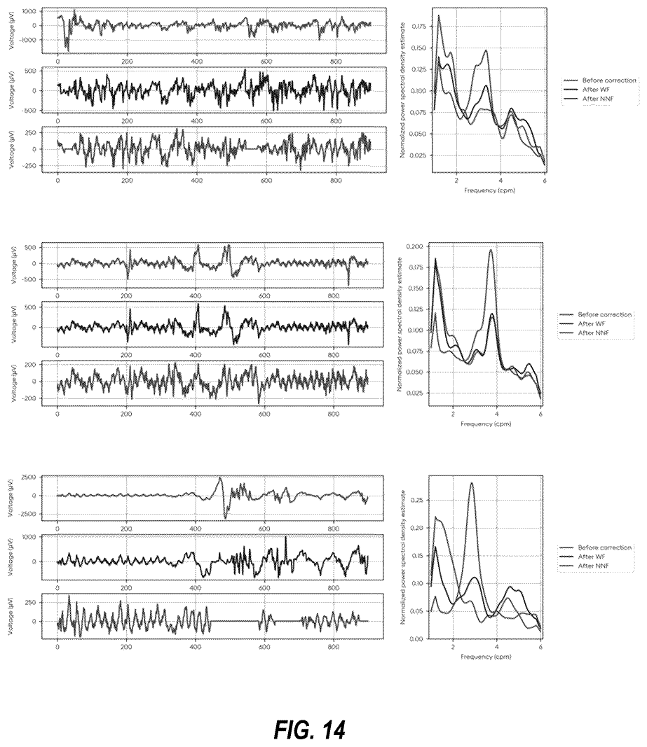

Visual inspection of the processed signals showed that the NNF has an enhanced ability to discern and correct complex artifacts while preserving the underlying physiological waveform. FIG. 14 illustrates three 15-minute segments of single channel BSGM recordings. Each panel contains the signals for a different segment before correction (top), after correction with WF (middle), and after correction with NNF (bottom), along with the normalized power spectral density estimates calculated using the Welch method and smoothed with a 0.5 cpm bandwidth moving average (right). Flat lines in the corrected signals indicate data removed by the filter.

FIG. 14 further provides representative examples of the system's performance on heavily contaminated signals. The power spectral densities illustrate how stable gastric signals can be overpowered by high-amplitude broadband artifacts. The increased concentration of activity in the plausible gastric frequency range for the NNF-processed data as compared to the WF demonstrates the improved reliability of the detection of stable gastric activity. These examples illustrate that the NNF can offer improvements in both the correction of artifacts (top and middle), and also in the removal of data that is not able to be corrected (bottom). In instances where the signals are severely corrupted by large artifacts extending across several minutes, the NNF more reliably removes the signal.

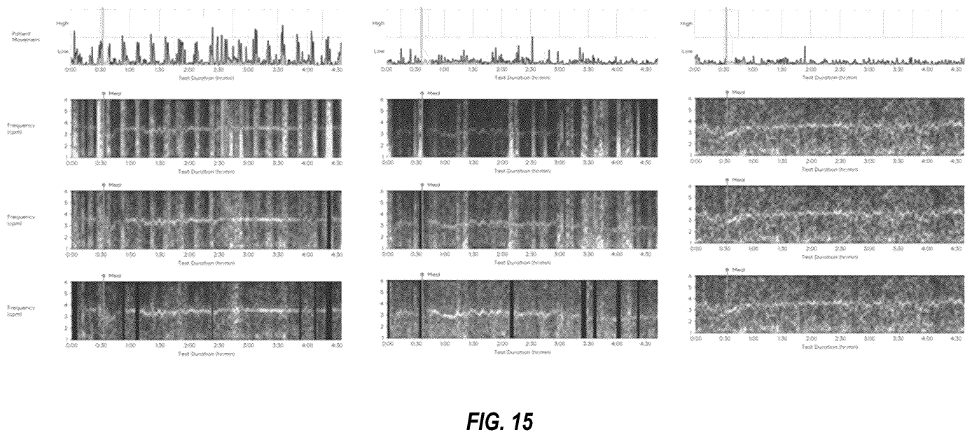

FIG. 15 illustrates spectrograms and accelerometer-based activity index for a research study where the patient was standing up every 15 minutes to perform a gastric emptying breath test (left), a clinical test from the testing set with significant artifacts (middle), and a clinical test from the testing set with clean signals throughout the duration of the test (right). For each test, spectrograms are generated using the complete Gastric Alimetry Algorithm v3.0.0 signal processing pipeline executed with no artifact correction (top), the WF (middle), and the NNF (bottom). Aside from the artifact correction module, there are no other modifications to the signal processing between the methods.

The challenges posed by movement-based artifacts for spectral analysis are evident in the spectrograms without any dedicated artifact correction, as the underlying gastric rhythm (horizontal band at ˜3 cpm) is obscured by vertical bands of high-power, broadband noise corresponding to periods of artifact. These vertical bands are only partially corrected/removed by the WF processing. The NNF-processed data reveals a clear, stable gastric frequency band. This clean spectral representation the basis for the accurate calculation of quantitative biomarkers such as the Principal Gastric Frequency (PGF) and the Gastric Alimetry Rhythm Index (GA-RI), which are further utilized for phenotyping gastric disorders. The final example demonstrates the NNF's ability to preserve the original signal when no artifacts are present.

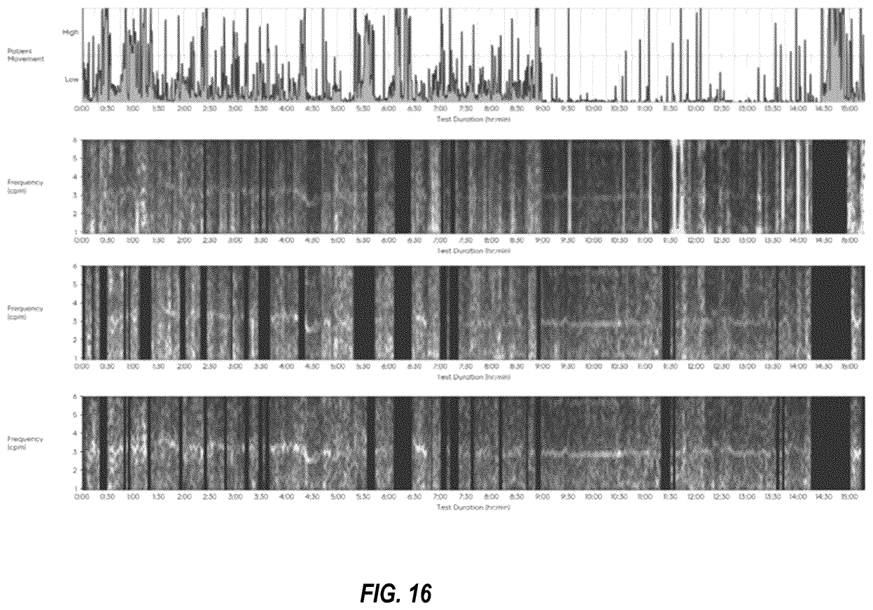

FIG. 16 illustrates spectrograms and accelerometer-based activity index for a 15-hour recording where the patient was not limited in their movement throughout the day. Spectrograms are generated using the complete Gastric Alimetry Algorithm v3.0.0 signal processing pipeline executed with no artifact correction (top), the WF (middle), and the NNF (bottom). Aside from the artifact correction module, there are no other modifications to the signal processing between the methods. FIG. 16 demonstrates that the NNF enables more reliable detection of gastric activity even in settings where patient activity is not monitored or controlled. As compared to the WF, the NNF removes less data, and where data is retained, activity is more concentrated in the gastric activity band.

While exemplary embodiments have been illustrated and described, it will be appreciated that various changes can be made therein without departing from the spirit and scope of the disclosure.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present, or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatuses are referred to as “lowest”, “best”, “minimum,” or the like. It will be appreciated that such descriptions are intended to indicate that a selection among many used functional alternatives can be made, and such selections need not be better, smaller, or otherwise preferable to other selections.

What is claimed is:

1. A method of processing gastric activity data comprising:
receiving electrical signals associated with gastric activity of a patient over a predetermined time period, wherein the electrical signals are measured with an electrode array patch disposed over an abdomen skin surface of the patient;
for each received signal:
providing the signal as an input signal to a correction neural network and a removal neural network; and
outputting, by the correction neural network, an intermediate corrected signal generated based on the input signal, wherein, in the intermediate corrected signal, each segment of the input signal identified by the correction neural network to contain noisy data is replaced with a corrected segment generated by the correction neural network; and
generating an initial report based on the intermediate corrected signal, wherein the initial report comprises a gastrointestinal phenotype based at least in part on the intermediate corrected signal.

2. The method of claim 1, further comprising:
outputting a confidence score for each segment of the input signal identified by the removal neural network to contain noisy data, wherein the confidence score represents a difference between the input signal and a predicted corrected signal;
determining whether each confidence score associated with a corrected segment is below a preconfigured threshold value;
in response to determining that the confidence score associated with the corrected segment is below the preconfigured threshold value, replacing, by a removal module, the corrected segment with a segment representing deleted data to generate a final corrected signal; and
generating a final report based on the final corrected signal, wherein the final report identifies the gastrointestinal phenotype based at least in part on the final corrected signal.

3. The method of claim 2, wherein the final report comprises a visual representation of the final corrected signal.

4. The method of claim 3, wherein the visual representation identifies data segments replaced by corrected segments.

5. The method of claim 3, wherein the visual representation identifies data segments replaced by segments representing deleted data.

6. The method of claim 1, wherein the gastrointestinal phenotype comprises a dysrhythmic phenotype, a high frequency phenotype, a low meal response phenotype, a sensorimotor phenotype, a continuous phenotype, and a delayed onset phenotype.

7. The method of claim 1, wherein the gastrointestinal phenotype comprises a low amplitude phenotype, a high frequency phenotype, a low frequency phenotype, or a delayed meal response phenotype.

8. The method of claim 2, further comprising:
determining one or more normalized biometrics over at least a predetermined time period from the final corrected signal;
correlating the one or more normalized biometrics and patient symptom information received over the predetermined time period;
determining a measure of correlation over the predetermined time period; and
determining the gastrointestinal phenotype based at least in part on the measure of correlation.

9. The method of claim 8, wherein the one or more normalized biometrics comprises at least one of a principal gastric frequency (PGF), a body mass index (BIM)-adjusted amplitude, Gastric Alimetry Rhythm Index (GA-RI), fed-fasted amplitude ratio (ff-AR), and a meal response ratio.

10. The method of claim 2, further comprising outputting a recommendation based at least in part on the gastrointestinal phenotype associated with the patient.

11. The method of claim 1, further comprising training the correction neural network, wherein training the correction neural network comprises:
providing an original set of patient data;
generating training data based on the original set of patient data; and
training a training module using the training data based on the original set of patient data.

12. The method of claim 11, further comprising:
generating synthetic data; and
training a training module using the training data based on the original set of patient data and the synthetic data.

13. The method of claim 11, wherein training the correction neural network comprises utilizing a loss function.

14. The method of claim 13, wherein the loss function utilized in training the correction neural network comprises a normalization operation such that error measurements between an estimated signal and a true signal are expressed proportionally to the magnitude of the clean signal.

15. The method of claim 13, wherein the loss function utilized in training the correction neural network comprises a pointwise scaling operation configured to increase the loss for errors occurring at time points where the true signal is less corrupted by noise.

16. The method of claim 13, wherein the loss function utilized in training the correction neural network comprises an adaptive scaling factor for each time point, the factor being inversely related to an estimate of noise present in the true signal at that time point.

17. The method of claim 11, further comprising identifying segments of the original set of patient data to be classified as clean data segments and noisy data segments.

18. The method of claim 12, wherein generating the synthetic data comprises synthesizing clean data segments and noisy data segments.

19. The method of claim 12, further comprising augmenting and recombining the clean data segments and the noisy data segments identified from the original set of patient data.

20. The method of claim 12, further comprising augmenting and recombining the synthesized clean data segments and the synthesized noisy data segments.

21. The method of claim 12, further comprising augmenting and recombining the synthesized clean data segments and the noisy data segments.

22. The method of claim 12, further comprising augmenting and recombining the noisy data segments and the clean data segments.

23. The method of claim 2, further comprising training the removal neural network based at least in part on the correction neural network.

24. A system for processing gastric activity data comprising:
an electrode array patch disposed over an abdomen skin surface of a patient for measuring electrical signals associated with gastric activity of the patient over a predetermined time period; and
a processor configured to:
receive electrical signals from the electrode array patch;
for each received signal:
provide the signal as an input signal to a correction neural network and a removal neural network; and
output, by the correction neural network, an intermediate corrected signal generated based on the input signal, wherein, in the intermediate corrected signal, each segment of the input signal identified by the correction neural network to contain noisy data is replaced with a corrected segment generated by the correction neural network; and
generate an initial report based on the intermediate corrected signal, wherein the initial report comprises a gastrointestinal phenotype based at least in part on the intermediate corrected signal.

25. A non-transitory computer-readable medium storing instructions executable by one or more processors for causing the one or more processors to perform operations comprising:
receiving electrical signals associated with gastric activity of a patient over a predetermined time period, wherein the electrical signals are measured with an electrode array patch disposed over an abdomen skin surface of the patient;
for each received signal:
providing the signal as an input signal to a correction neural network and a removal neural network; and
outputting, by the correction neural network, an intermediate corrected signal generated based on the input signal, wherein, in the intermediate corrected signal, each segment of the input signal identified by the correction neural network to contain noisy data is replaced with a corrected segment generated by the correction neural network;
generating an initial report based on the intermediate corrected signal, wherein the initial report comprises a gastrointestinal phenotype based at least in part on the intermediate corrected signal;
outputting a confidence score for each segment of the input signal identified by the removal neural network to contain noisy data, wherein the confidence score represents a difference between the input signal and a predicted corrected signal;
determining whether each confidence score associated with a corrected segment is below a preconfigured threshold value;
in response to determining that the confidence score associated with the corrected segment is below the preconfigured threshold value, replace, by a removal module, the corrected segment with a segment representing deleted data to generate a final corrected signal; and
generating a final report based on the final corrected signal, wherein the final report identifies the gastrointestinal phenotype based at least in part on the final corrected signal.
